(19) United States
(12) Patent Application Publication
(10) Pub. No.: US 2017/0296627 A1
(43) Pub. Date: Oct. 19, 2017

(54) EXERSOMES, METHODS OF PRODUCING AND METHOD OF USING

(71) Applicant: EXERKINE CORPORATION, Hamilton (CA)

(72) Inventor: Mark TARNOPOLSKY, Hamilton (CA)

(21) Appl. No.: 15/449,599

(22) Filed: Mar. 3, 2017

Related U.S. Application Data
(63) Continuation-in-part of application No. PCT/CA2015/050854, filed on Sep. 4, 2015.
(60) Provisional application No. 62/211,312, filed on Aug. 28, 2015.
(60) Provisional application No. 62/112,940, filed on Feb. 6, 2015.
(60) Provisional application No. 62/046,518, filed on Sep. 5, 2014.

(51) Int. Cl. A61K 38/22; A61K 9/16

(52) U.S. Cl. CPC A61K 38/22; A61K 9/16

(57) ABSTRACT

An exosome pellet or physiological solution comprising resuspended exosomes is provided. The exosomes are essentially free from undesirable particles having a diameter less than 20 nm or greater than 140 nm, and the exosomes comprise one or more metabolic products. The exosomes may be used to induce mitochondrial biogenesis, increase thermogenesis (browning) of subcutaneous white adipose tissue, and/or mediate other systemic effects of exercise in a mammal.

### FIELD OF THE INVENTION

The present invention relates to exosomes and novel methods for producing and using exosomes.

### BACKGROUND OF THE INVENTION

Physical inactivity is a major threat to public health in Canada, and is a modifiable risk factor for metabolic diseases (type 2 diabetes, obesity) and other chronic diseases including muscle atrophy (secondary to aging called sarcopenia, cancer cachexia, disuse atrophy, and/or bed rest/immobilization associated atrophy), cardiovascular diseases, degenerative disorders (Alzheimer's Disease, Parkinson's Disease, and Huntington's Disease), and neuromuscular disorders. Current therapies for these pathologies are only moderately helpful in managing the disease and primarily address the secondary symptoms rather than the pathology itself. For example, current therapies for type 2 diabetes are helpful, however they remain inadequate in preventing the negative effects of type 2 diabetes and metabolic syndrome on the cardiovascular system, cancer and other aging-associated co-morbidities.

Studies have demonstrated that the benefits of endurance exercise are systemic in nature and counteract diseases of aging (sarcopenia, cardiovascular disease, dementia, cataract, osteoporosis, infertility), mitochondrial disease, and the metabolic syndrome (type 2 diabetes, insulin resistance, obesity). While skeletal muscle and cardiovascular function are often the primary focus of exercise interventions, there is evidence that endurance exercise has widespread systemic effects and confers cellular and phenotypic protection in multiple organs and tissues. However, the biological mediators of the multi-systemic benefits conferred by physical activity have not been fully elucidated.

It would be desirable to develop an understanding of these biological mediators in order to develop therapeutic strategies that target one or more metabolic-related pathologies or other chronic diseases.

### SUMMARY OF THE INVENTION

It has now been determined that exosomes obtained from a biological source and comprising one or more metabolic products are useful to induce mitochondrial biogenesis, increase thermogenesis (browning/beiging) of subcutaneous white adipose tissue, and/or mediate other systemic effects of exercise. Induction of mitochondrial biogenesis, increasing thermogenesis (browning/beiging) of subcutaneous white adipose tissue, and/or mediating other systemic effects of exercise is useful to treat, for example, metabolic syndromes, aging associated disorders, neurological disorders and neuromuscular disorders.

Thus, in one aspect of the invention, an exosome pellet or physiological solution comprising resuspended exosome pellet is provided. The pellet or solution comprises exosomes essentially free from particles having a diameter less than 20 or greater than 140 nm, and the exosomes comprise one or more metabolic products.

In one embodiment, the exosomes are obtained from a biological sample in which the exosomes are loaded with one or more endogenous metabolic products.

In another embodiment, the exosomes are engineered to include one or more exogenous metabolic products.

In another aspect, a method of inducing mitochondrial biogenesis in a mammal is provided. The method comprises the steps of administering to the mammal a physiological solution comprising exosomes which is essentially free from particles having a diameter less than 20 or greater than 140 nm, wherein the exosomes comprise one or more metabolic products.

These and other aspects of the invention will be described by reference to the following figures.

### BRIEF DESCRIPTION OF THE FIGURES

FIG. 1 illustrates the results of exosomal isolation from serum using a novel isolation methodology;

FIG. 2 graphically shows that acute endurance exercise increases serum exosomal content in mice;

FIG. 3 shows that exosome-containing serum from athletes carries exercise-induced proliferative factors for optimal maintenance and rejuvenation of human dermal fibroblasts (A/B);

FIG. 4 graphically illustrates that proliferative factors in serum from exercising mammals are stored in exersomes;

FIG. 5 graphically illustrates that exosomes isolated from endurance exercise-trained mice (END) increases basal voluntary endurance activity in sedentary mice (B) as compared to the effect of exosomes from sedentary mice (A), as shown by an overlay of the results (C);

FIG. 6 graphically illustrates that exersomes increase basal voluntary endurance activity in sedentary mice (B) as compared to endurance exercise-trained mice (A), as shown in an overlay of voluntary wheel activity of both groups (C);

FIG. 7 graphically illustrates that exersomes from exercise-trained mice (END) increase maximum endurance capacity of sedentary mice (B) to a level comparable to endurance exercise-trained mice (A);

FIG. 8 graphically illustrates that administration of END exosomes results in an increase in basal voluntary endurance activity in high-fat fed mouse model of obesity and type 2 diabetes (B) as compared to the activity resulting with administration of SED exosomes (A), as shown in an overlay of the results (C);

FIG. 9 graphically illustrates that END exosomes result in increased maximum endurance capacity as compared to SED exosomes in high-fat fed mouse model of obesity and type 2 diabetes;

FIG. 10 illustrates that SED exosomes protect high-fat fed mice against diet-induced obesity and diabetes as shown by effects on body weight (A) and glucose tolerance (B);

FIG. 11 illustrates that basal voluntary endurance activity in an mtDNA mutator mouse model of aging and mitochondrial dysfunction is increased on treatment with END exosomes (B) as compared to treatment with SED exosomes (A) as shown in an overlay (C) of the results;

FIG. 12 illustrates that END exosomes increase maximum endurance capacity of mtDNA mutator mouse model of aging and mitochondrial dysfunction;

FIG. 13 illustrates that endurance exercise promotes cross-talk between skeletal muscle and various organs/tissues as well as inter-organ/tissue cross-talk;

FIG. 14 graphically compares the PGC-1α-mediated mitochondrial biogenesis gene signature in skeletal muscle of sedentary and exercised mice, untreated and treated with exosomes from sedentary and exercised mice;

FIG. 15 graphically compares the effect on mtDNA copy number in muscle harvested from mice treated with exosomes from sedentary and exercised mice (A), from mice treated with an exosome inhibitor (B), and in mice treated with both (C);

FIG. 16 graphically illustrates induction of the PGC-1α-mediated mitochondrial biogenesis gene signature (A) and a systemic increase in mitochondrial cytochrome c oxidase activity (B) in PolG-WT mice treated with exersomes;

FIG. 17 graphically illustrates that exersomes from mice subjected to acute endurance exercise exhibited induced beige fat gene expression;

FIG. 18 illustrates the proteomic (A) and genomic (B) study of exersome content;

FIG. 19 illustrates the presence of METRNL in exosomes from MCK-PGC-1a mice (A), and graphically compares body weight (B), fasting insulin (C) and glucose tolerance (D) in exercised mice vs. mice treated with METRNL;

FIG. 20 graphically compares the effect of METRNL and exercise (A/B) on beige fat gene expression in primary human subcutaneous pre-adipocytes;

FIG. 21 graphically illustrates that fndc5 gene is induced in response to transgenic over-expression of PGC-1a in muscle (A), in response to endurance exercise training in mice (B) and in humans (C); and

FIG. 22 graphically illustrates the ability of FNDC5 to induce the browning gene expression program in pre-adipocytes.

### DETAILED DESCRIPTION OF THE INVENTION

In one aspect, the present invention relates to exosomes comprising one or more metabolic products rendering them useful to induce mitochondrial biogenesis, increase thermogenesis (browning) of subcutaneous white adipose tissue, and/or mediate other systemic effects of exercise, in a mammal.

The term “exosome” refers to cell-derived vesicles having a diameter of between about 40 and 140 nm, preferably a diameter of about 40-120 nm, for example, a diameter of about 40 nm, 50 nm, 60 nm, 70 nm, 80 nm, 90 nm, 100 or 120 nm. Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g. immature dendritic cells (wild-type or immortalized), induced and non-induced pluripotent stem cells, fibroblasts, platelets, immune cells, reticulocytes, tumour cells, mesenchymal stem cells, satellite cells, hematopoietic stem cells, pancreatic stern cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g. CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). As used herein, the term “mammal” is meant to encompass, without limitation, humans, domestic animals such as dogs, cats, horses, cattle, swine, sheep, goats and the like, as well as non-domesticated animals such as, but not limited to, mice, rats and rabbits. Exosomes may also be obtained from a non-mammal or from cultured non-mammalian cells. As the molecular machinery involved in exo some biogenesis is believed to be evolutionarily conserved, exosomes from non-mammalian sources include surface markers which are isoforms of mammalian surface markers, such as isoforms of CD9 and CD63, which distinguish them from other cellular vesicles. The term “non-mammal” is meant to encompass, for example, exosomes from microorganisms such as bacteria, flies, worms, plants, fruit/vegetables (e.g. corn, pomegranate) and yeast.

In one embodiment, the exosomes of interest, comprising one or more metabolic products, are isolated from a suitable biological sample obtained from a mammal that regularly performs exercise, immediately following exercise, or up to (and including) 4 hours post-exercise. Exosomes isolated from an exercising mammal are herein referred to as “exersomes” or metabolically-induced exosomes. The term “exercise” is meant to encompass endurance exercise, high-intensity interval training, resistance exercise, and the like, e.g. exercise that achieves a level of working of at least about 3-6 metabolic equivalents (METS), and combinations thereof (e.g. any combination of endurance exercise, high-intensity interval exercise, or >50% of the one repetition maximum (resistance exercise)). METS is the energy expenditure of a physical activity or exercise defined as the ratio of the metabolic rate of an exercising individual (and therefore the rate of energy consumption) during a specific physical activity to a reference basal metabolic rate. Regularly performing exercise refers to the performance of exercise for a duration of at least a month, at a frequency of at least 2 days/week, and preferably at least 3 or more days a week, for a period of at least 30 consecutive minutes per day, preferably 45 minutes or greater, such as 60 minutes or greater, or 75-90 minutes or more. Exercise may include endurance activities such as brisk walking, jogging, running, dancing, swimming, bicycling, sports, interval training, resistance exercise, and the like. Interval training refers to repetitive bouts of exercise that may be at high or lower intensities provided it meets minimal METS requirements. High intensity interval training would include activities such as sprints (e.g. 10 second to 4 minute sprints) followed by a recovery time (e.g. of 10 seconds to 4 minutes). The term “resistance exercise” refers to weight training or other resistance exercise (plyometrics, hydraulic machines, etc.) with a resistance at least 50% of the one repetition maximum, performed in sets of repetitions (for example, 8-15 repetitions), followed by a recovery between sets, for a period of time sufficient to achieve minimal METS requirements. One repetition maximum is the maximal voluntary contraction strength for a single movement where a second movement is impossible. An increase in the amount (intensity and/or duration) of exercise performed will increase the concentration of exersomes in the blood.

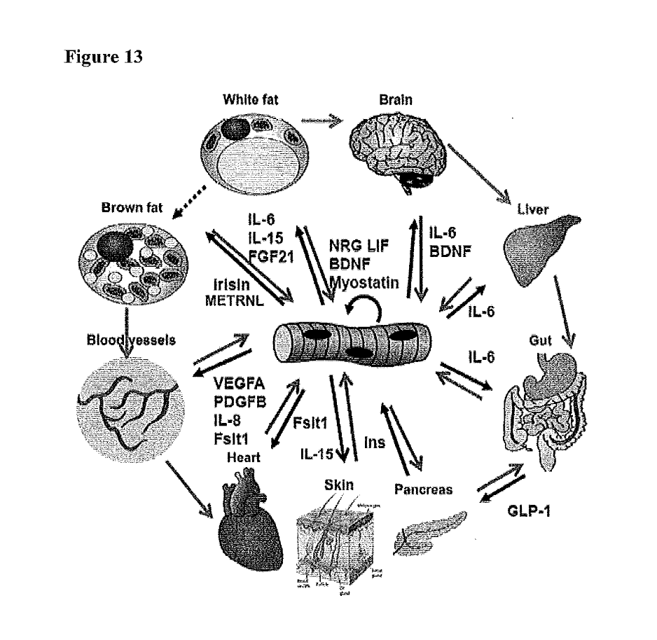

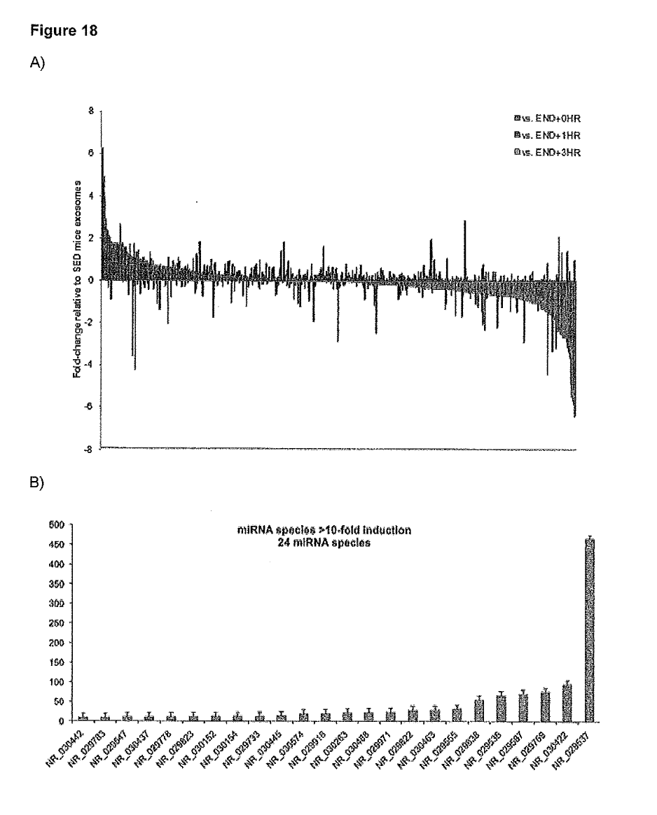

Exersomes according to the present invention have been determined to incorporate metabolic products that result from exercise (metabolically induced products) and which are useful to induce mitochondrial biogenesis, increase thermogenesis (browning) of subcutaneous white adipose tissue, and/or mediate other systemic effects of exercise, either alone or in combination. Examples of such metabolic products include, but are not limited to, microRNA (miRNA), messenger RNA (mRNA), cytokines such as chemokines, interleukins and lymphokines, and other proteins such as growth factors and the like. FIG. 13 exemplifies events and products arising from exercise. Examples of proteins incorporated within exersomes (metabolically induced proteins) include, but are not limited to, Platelet-derived growth factor subunit B (PDGF-B), Meteorin-like protein (METRNL), Fibronectin type III domain-containing protein 5 (FNDC5), Fibronectin type III domain-containing protein 4 (FNDC4), Shisa family member 5 (Shisa5), secreted phosphoprotein 1 (SPP1), Prolactin-inducible protein (PIP), Tropomyosin alpha-1 (TPM1), Prosaposin or Proactivator polypeptide (PSAP), and Vascular endothelial growth factor B (VEGF-B). Examples of miRNA incorporated within exersomes (metabolically induced miRNA), include but are not limited to, miR-677, miR-107, miR-133a-1, miR-496, miR-101b, miR-128-2, miR-469, miR-471, miR-15a, miR-679, miR-504, miR-411, miR-541, miR-707, miR-451, miR-125b-1, miR-690, miR-142, miR-219-2, miR-99b, miR-200b, miR-340, miR-551b and miR-101a, as shown in FIG. 18B. Metabolically induced products, such as proteins and miRNA, are present in exersomes in an amount which is greater than the amount of these products in non-metabolically-induced exosomes, by at least about 2-fold or greater, e.g. 5-fold to 10-fold or greater.

Exersomes may be obtained from the appropriate mammalian biological sample, e.g. blood or other sample as set out above, using a combination of isolation techniques, for example, centrifugation, filtration, ultracentrifugation and PEG-based methodologies. In one embodiment, exosomes may be isolated from a biological sample using a method including the steps of: i) optionally exposing the biological sample to a first centrifugation to remove cellular debris greater than about 7-10 microns in size from the sample and obtaining the supernatant following centrifugation; ii) optionally subjecting the supernatant from step i) to centrifugation to remove microvesicles and apoptotic bodies therefrom; iii) optionally microfiltering the supernatant from step ii) and collecting the microfiltered supernatant; iv) combining the microfiltered supernatant from step iii) with a polyethylene glycol solution to precipitate the exosomes and subjecting the solution to at least one round of centrifugation to obtain an exosome pellet; and v) re-suspending the exosome pellet from step iv) in a trehalose solution and conducting an optional centrifugation step to remove vesicles having a diameter of greater than 140 nm from the solution.

In accordance with an aspect of the present invention, the process of isolating exosomes from a biological sample includes a first optional step of removing undesired large cellular debris from the sample, i.e. cells, cell components, apoptotic bodies and the like greater than about 7-10 microns in size. This first step is generally conducted by centrifugation, for example, at 1000-4000×g for 10 to 60 minutes at 4° C., preferably at 1500-2500×g, e.g. 2000×g, for a selected period of time such as 10-30 minutes, 12-28 minutes, 14-24 minutes, 15-20 minutes or 16, 17, 18 or 19 minutes. As one of skill in the art will appreciate, a suitable commercially available laboratory centrifuge, e.g. Thermo-Scientific™ or Cole-Parmer™, is employed to conduct this isolation step. To enhance exosome isolation, the resulting supernatant is subjected to an additional optional centrifugation step to further remove cellular debris and apoptotic bodies, such as debris that is at least about 7-10 microns in size, by repeating this first step of the process, i.e. centrifugation at 1000-4000×g for 10 to 60 minutes at 4° C., preferably at 1500-2500×g, e.g. 2000×g, for the selected period of time.

Following removal of cell debris, the supernatant resulting from the first centrifugation step(s) is separated from the debris-containing pellet (by decanting or pipetting it off) and may then be subjected to an optional additional (second) centrifugation step, including spinning at 12,000-15,000×g for 30-90 minutes at 4° C. to remove intermediate-sized debris, e.g. debris that is greater than 6 microns size. In one embodiment, this centrifugation step is conducted at 14,000×g for 1 hour at 4° C. The resulting supernatant is again separated from the debris-containing pellet.

The resulting supernatant is collected and subjected to a third optional centrifugation step, including spinning at between 40,000-60,000×g for 30-90 minutes at 4° C. to further remove impurities such as medium to small-sized microvesicles greater than 0.3 microns in size e.g. in the range of about 0.3-6 microns. In one embodiment, the centrifugation step is conducted at 50,000×g for 1 hour. The resulting supernatant is separated from the pellet for further processing.

The supernatant is then optionally filtered to remove debris, such as bacteria and larger microvesicles, having a size of about 0.22 microns or greater, e.g. using microfiltration. The filtration may be conducted by one or more passes through filters of the same size, for example, a 0.22 micron filter. Alternatively, filtration using 2 or more filters may be conducted, using filters of the same or of decreasing sizes, e.g. one or more passes through a 40-50 micron filter, one or more passes through a 20-30 micron filter, one or more passes through a 10-20 micron filter, one or more passes through a 0.22-10 micron filter, etc. Suitable filters for use in this step include the use of 0.45 and 0.22 micron filters.

The microfiltered supernatant (filtrate) may then be combined with a polyethylene glycol (PEG) solution to precipitate exosomes within the filtrate. As would be appreciated by one of skill in the art, a variety of PEG formulations may be used. Preferably, these formulations comprise PEG chain lengths having an average molecular weight of between about 400 to 20,000 daltons (e.g. 1000 to 10,000 daltons, such as 6000 daltons). Similarly, the exosome-PEG solutions may have varying final concentrations of PEG, for example, a final concentration of PEG may be between about 5-15% (such as 8%). Preferably, the filtrate is combined with an equal volume of the PEG solution, having a strength in the range of about 10-20% PEG. Salts may be added to the PEG solution to enhance the precipitation of exosomes. Preferably, a salt such as NaCl is added to the PEG solution so that the final concentration of salt in the exosome-PEG-salt solution is between about 50 to 1,000 mM (such as 500 mM). The PEG-filtrate is gently mixed and incubated under conditions suitable for exosome precipitation, e.g. incubated for 30 minutes at 4° C. Some samples may require a longer incubation period for exosome precipitation to occur.

Following incubation, the precipitated exosomes were pelleted by centrifugation, e.g. at 10,000×g for 10 min at 4° C., and the pellet was solubilized in a suitable saccharide solution, such as a trehalose solution, that is effective to reduce aggregation of the exosomes. The saccharide is preferably solubilized in a physiological buffer, such as saline or PBS. In one embodiment, a trehalose solution of various concentrations is effective at reducing the aggregation of exosomes, such as a trehalose concentration between 10 mM to 1,000 mM (e.g. 500 mM).

To remove non-exosome extracellular vesicles (i.e. vesicles larger than 140 nm), the trehalose exosome solution may be subjected to further optional centrifugation or ultracentrifugation steps, for example, at 15,000×g-150,000×g for 1 hr at 4° C. If ultracentrifugation is performed, exosomes will be present in both the resultant pellet and supernatant fractions, generally with a larger quantity of exosomes in the supernatant.

To enhance removal of impurities that are smaller than the exosomes, e.g. smaller than 20 nm, the exosome-trehalose solution may be subjected to an optional ultrafiltration step using either a direct-flow filtration technique (such as a centrifugal spin filter) or a cross-flow filtration technique (such as a tangential flow system). As would be appreciated by one of skill in the art, filtration membranes suitable for this step may possess a molecular weight cut-off (MWCO) rating in the range of 3-500 kDa and preferably between 100-300 kDa.

In another embodiment, exosome isolation may include the steps of: i) exposing the biological sample to a first centrifugation to remove cellular debris greater than about 7-10 microns in size from the sample and obtaining the supernatant following centrifugation; ii) subjecting the supernatant from step i) to centrifugation to remove microvesicles and apoptotic bodies therefrom; iii) microfiltering the supernatant from step ii) and collecting the microfiltered supernatant; iv) subjecting the microfiltered supernatant from step iii) to at least one round of ultracentrifugation to obtain an exosome pellet; and v) re-suspending the exosome pellet from step iv) in a physiological solution and conducting a second ultracentrifugation in a density gradient and remove the exosome pellet fraction therefrom.

The centrifugation and filtration steps (steps i)-iii)) are as previously described.

Following the initial centrifugation and filtration steps, the exosomal solution is then subjected to ultracentrifugation to pellet exosomes and any remaining contaminating microvesicles (between 100-220 nm). This ultracentrifugation step is conducted at 110,000-170,000×g for 1-3 hours at 4° C., for example, 170,000×g for 3 hours. This ultracentrifugation step may optionally be repeated, e.g. 2 or more times, in order to enhance results. Any commercially available ultracentrifuge, e.g. Thermo-Scientific™ or Beckman™, may be employed to conduct this step. The exosome-containing pellet is removed from the supernatant using established techniques and re-suspended in a suitable physiological solution.

Following ultracentrifugation, the re-suspended exosome-containing pellet is subjected to density gradient separation to separate contaminating microvesicles from exosomes based on their density. Various density gradients may be used, including, for example, a sucrose gradient, a colloidal silica density gradient, an iodixanol gradient, or any other density gradient sufficient to separate exosomes from contaminating microvesicles (e.g. a density gradient that functions similar to the 1.100-1.200 g/ml sucrose fraction of a sucrose gradient). Thus, examples of density gradients include the use of a 0.25-2.5 M continuous sucrose density gradient separation, e.g. sucrose cushion centrifugation, comprising 20-50% sucrose; a colloidal silica density gradient, e.g. Percoll™ gradient separation (colloidal silica particles of 15-30 nm diameter, e.g. 30%/70% w/w in water (free of RNase and DNase), which have been coated with polyvinylpyrrolidone (PVP)); and an iodixanol gradient, e.g. 6-18% iodixanol. The resuspended exosome solution is added to the selected gradient and subjected to ultracentrifugation at a speed between 110,000-170,000×g for 1-3 hours. The resulting exosome pellet is removed and re-suspended in physiological solution.

Depending on the density gradient used, the re-suspended exosome pellet resulting from the density gradient separation may be ready for use. For example, if the density gradient used is a sucrose gradient, the appropriate sucrose fractions are collected and may be combined with other collected sucrose fractions, and the resuspended exosome pellet is ready for use, or may preferably be subjected to an ultracentrifugation wash step at a speed of 110,000-170,000×g for 1-3 hours at 4° C. If the density gradient used is, for example, a colloidal silica (Percoll™) or an iodixanol density gradient, then the resuspended exosome pellet may be subjected to additional wash steps, e.g. subjected to one to three ultracentrifugation steps at a speed of 110,000-170,000×g for 1-3 hours each at 4° C., to yield an essentially pure exosome-containing pellet. The pellet is removed from the supernatant and may be re-suspended in a physiologically acceptable solution for use.

As one of skill in the art will appreciate, the exosome pellet from any of the centrifugation or ultracentrifugation steps may be washed between centrifugation steps using an appropriate physiological solution, e.g. sterile PBS, sterile 0.9% saline or sterile carbohydrate-containing 0.9% saline buffer.

The present methods advantageously provide a means to obtain mammalian and non-mammalian exosomes which are useful therapeutically. In some embodiments, the methods yield exosomes which exhibit a high degree of purity, for example, at least about 50% pure, and preferably, at least about 60%, 70%, 80%, 90% or 95% or greater pure. Preferably, the exosomes are “essentially free” from cellular debris, apoptotic bodies and microvesicles having a diameter less than 20 nm or greater than 140 nm, and preferably less than 40 nm or greater than 120 nm, and which are biologically intact, e.g. not clumped or in aggregate form, and not sheared, leaky or otherwise damaged. Exosomes isolated according to the methods described herein exhibit a degree of stability, that may be evidenced by the zeta potential of a mixture/solution of such exosomes, for example, a zeta potential of at least a magnitude of ±10 mV, e.g. ≦−10 or ≧+10, and preferably, a magnitude of at least 20 mV, 30 mV, 40 mV, 50 mV, 60 mV, 70 mV, 80 my, or greater. The term “zeta potential” refers to the electrokinetic potential of a colloidal dispersion, and the magnitude of the zeta potential indicates the degree of electrostatic repulsion between adjacent, similarly charged particles (exosomes) in a dispersion. For exosomes, generally the higher the magnitude of the zeta potential, the greater the stability of the exosomes.

Moreover, high quantities of exosomes are achievable by the present isolation method. With the PEG-based method, 1 mL of serum yields about 5-10 mg of protein. With the ultracentrifugation/density gradient method, 1 mL of serum or 15-20 mL of cell culture spent media (from at least about 2×10⁶ cells) yields about 100-2000 total protein. Thus, solutions comprising exosomes at a concentration of at least about 5 μg/μL, and preferably at least about 10-25 mg/μL, may readily be prepared due to the high exosome yields obtained by the present method. The term “about” as used herein with respect to any given value refers to a deviation from that value of up to 10%, either up to 10% greater, or up to 10% less.

Exosomes isolated in accordance with the methods herein described, beneficially retaining integrity, and exhibiting purity (being “essentially free” from undesirable entities having a diameter less than 20 nm and or greater than 140 nm), stability and biological activity both in vitro and in vivo, have not previously been achieved. Thus, the present exosomes are uniquely useful, for example, diagnostically and/or therapeutically. They have also been determined to be non-allergenic, and thus, safe for autologous, allogenic, and xenogenic use.

Exersomes obtained using the present method may be formulated for therapeutic use by combination with a pharmaceutically or physiologically acceptable carrier. The expressions “pharmaceutically acceptable” or “physiologically acceptable” means acceptable for use in the pharmaceutical and veterinary arts, i.e. not being unacceptably toxic or otherwise unsuitable for physiological use. As one of skill in the art will appreciate, the selected carrier will vary with intended utility of the exersome formulation. In one embodiment, exersomes are formulated for administration by infusion or injection, e.g. subcutaneously, intraperitoneally, intramuscularly or intravenously, and thus, are formulated as a suspension in a medical-grade, physiologically acceptable carrier, such as an aqueous solution in sterile and pyrogen-free form, optionally, buffered or made isotonic. The carrier may be distilled water (DNase- and RNase-free), a carbohydrate-containing solution (e.g. sucrose or dextrose) or a saline solution comprising sodium chloride and optionally buffered. Suitable saline solutions may include varying concentrations of sodium chloride, for example, normal saline (0.9%), half-normal saline (0.45%), quarter-normal saline (0.22%), and solutions comprising greater amounts of sodium chloride (e.g. 3%-7%, or greater). Saline solutions may optionally include additional components, e.g. carbohydrates such as dextrose and the like. Examples of saline solutions including additional components, include Ringer's solution, e.g. lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In other embodiments, exersomes are formulated for administration by routes including, but not limited to, oral, intranasal, enteral, topical, sublingual, intra-arterial, intramedullary, intrathecal, inhalation, ocular, transdermal, vaginal or rectal routes, and will include appropriate carriers in each case. For example, exersome compositions for topical application may be prepared including appropriate carriers. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents, anti-oxidants and other preservatives may be added to the composition to prevent microbial growth and/or degradation over prolonged storage periods.

Alternatively, the exersome pellet may be stored for later use, for example, in cold storage at 4° C., in frozen form or in lyophilized form, prepared using well-established protocols. The exersome pellet may be stored in any physiological acceptable carrier, optionally including cryogenic stability and/or vitrification agents (e.g. DMSO, glycerol, trehalose, polyhydroxylated alcohols (e.g. methoxylated glycerol, propylene glycol), M22 and the like).

Isolated exersomes, according to an aspect of the present invention, are useful to induce mitochondrial biogenesis, increase thermogenesis (browning) of subcutaneous white adipose tissue, and/or mediate other systemic effects of exercise in a mammal, such as to reduce local and systemic inflammation, promote cellular redox balance, maintain optimal levels of all types of autophagy (micro and macro-autophagy, and chaperone-mediated autophagy), preserve proliferation and differentiation of stem cell populations (pluripotent stem cells, satellite cells, hematopoietic stem cells, and other stern cells that lead to formation of mammalian cells), increase organismal fecundity, and prevent multisystem decline with aging (e.g. sarcopenia, brain atrophy, gonadal atrophy, aged skin, graying of hair, etc.). Accordingly, exersomes are useful in a method of treating metabolic syndrome, diseases of mitochondrial etiology, neuromuscular and neurometabolic diseases, and other aging-associated comorbidities (e.g., cancer, dementia, cardiovascular diseases, cataracts, anemia, infertility etc.) in a mammal. The terms “treat”, “treating” and “treatment” are used broadly herein to denote methods that favorably alter the targeted disorder, including those that at least moderate or reverse the progression of, reduce the severity of, or prevent the disorder.

Thus, for use to induce mitochondrial biogenesis, increase thermogenesis (browning) of subcutaneous white adipose tissue, and/or mediate other systemic effects of exercise, a therapeutically effective amount of exersomes is administered to a mammal. The term “therapeutically effective amount” is an amount of exersome required to increase mitochondrial biogenesis, increase thermogenesis (browning) of subcutaneous white adipose tissue, and/or to mediate other systemic effects of exercise, while not exceeding an amount that may cause significant adverse effects. It is noted that exersomes isolated from the mammal being treated may be utilized, or alternatively, exersomes isolated from a different (or second) mammal may be used to treat a first mammal, e.g. autologous, allogenic, and xenogenic exersomes may be used in the treatment. Exersome dosages that are therapeutically effective will vary on many factors including the nature of the condition to be treated as well as the particular individual being treated. Appropriate exersome dosages for use include dosages sufficient to increase exersome plasma levels in a mammal being treated by at least about 10% of the exersome resting plasma level in the mammal being treated, for example, a dosage that mimics the increase in exersome content in an exercising mammal, or that mimics an increase in one or more of the metabolic products by about 10% of the metabolic product resting plasma level in the mammal being treated. The method includes administration of the selected dosage at a frequency of about 2-7 times a week to increase exersome content or targeted metabolic product plasma levels in a mammal being treated.

A method of inducing mitochondrial biogenesis, increasing thermogenesis (browning) of subcutaneous white adipose tissue, and/or mediating other systemic effects of exercise in a mammal is useful, for example, to treat metabolic syndrome in a mammal. The term “metabolic syndrome” is used herein to encompass disorders resulting from local and systemic mitochondrial dysfunction, including but not limited to, obesity, metabolic syndrome, type 2 diabetes, non-alcoholic fatty liver disease, hyperinsulinemia, hypoinsulinemia, hypertension, hyperhepatosteatosis, hyperuricemia, fatty liver, polycystic ovarian syndrome, hyperphagia, acanthosis nigricans, endocrine abnormalities, triglyceride storage disease, Bardet-Biedl syndrome, Lawrence-Moon syndrome, Parder-Labhart-Willi syndrome, primary mitochondrial genetic disorders (for example, MELAS, MERRF, LHON, POLG1 mutations, CPEO and the like), neurological disease (Parkinson disease, Alzheimer disease, ALS, muscular dystrophy and congenital myopathies), and aging-associated pathologies such as muscle degeneration and mass loss (sarcopenia), hearing loss (presbycusis), osteoporosis, macular degeneration, cognitive decline (senile dementia), anemia, hair loss or greying hair, aging and degeneration of skin, and glucose associated metabolic dysfunction (dysglycemia/type 2 diabetes).

In another aspect of the present invention, non-metabolically induced exosomes may be isolated from cultured cells and engineered to incorporate one or more exogenous metabolic products as cargo for use to induce mitochondrial biogenesis, increase thermogenesis (browning) of subcutaneous white adipose tissue, and/or mediate other systemic effects of exercise in a mammal, for example, metabolically-induced proteins such as, but not limited to, PDGF-B, METRNL, FNDC5, FNDC4, Shisa5, SPP1, PIP, TPM1, PSAP, and VEGF-B; mRNA encoding one or more metabolically-induced proteins, metabolically induced mRNA and metabolically-induced miRNA species. The term “exogenous” refers to metabolic products of a form or from a source that do not exist naturally in exosomes. As one of skill in the art will appreciate, functionally equivalent forms of any of these metabolic products may also be used, for example, any mammalian form of the product may be used, including human forms and functionally equivalent forms from other species such as mouse, rat, dog, cat, horse, cow, and the like, isoforms and variants, recombinantly produced forms or artificially modified forms, i.e. including modifications that do not adversely affect activity. The term “functionally equivalent” refers to a corresponding protein (including all isoforms, variants or modified versions of these proteins), mRNA (including all transcript variants), or miRNA that exhibit the same or similar activity (at least about 30% of the activity of the human form), or an mRNA that encodes a corresponding protein. Artificial modifications may include one or more amino acid substitutions (for example with a similarly charged amino acid), additions or deletions in a metabolic protein, or one or more base changes in an RNA species, Such modifications may be made to the protein or RNA species in order to render the metabolic product more suitable for therapeutic use, e.g. to increase stability and/or activity (such as fusion products, e.g. with Fc peptide). Suitable modifications will generally maintain at least about 70% sequence similarity with the active site and other conserved domains of native metabolic product, and preferably at least about 80%, 90%, 95% or greater sequence similarity.

Metabolic products may be introduced into exosomes using methods established in the art for introduction of cargo into cells. Thus, cargo may be introduced into exosomes, for example, using electroporation applying voltages in the range of about 20-1000 V/cm. Transfection using cationic lipid-based transfection reagents may also be used to introduce cargo into exosomes. Examples of suitable transfection reagents include, but are not limited to, Lipofectamine® MessengerMAX™ Transfection Reagent, Lipofectamine® RNAiMAX Transfection Reagent, Lipofectamine® 3000 Transfection Reagent, or Lipofectamine® LTX Reagent with PLUS™ Reagent. For cargo loading, a suitable amount of transfection reagent is used and may vary with the reagent, the sample and the cargo. For example, using Lipofectamine® MessengerMAX™ Transfection Reagent, an amount in the range of about 0.15 uL to 10 uL may be used to load 100 ng to 2500 ng mRNA or protein into exosomes. Other methods may also be used to load protein into exosomes including, for example, the use of cell-penetrating peptides.

Generally, exosomes are loaded with an amount of one or more metabolic products that renders a given dosage of loaded exosomes useful to induce mitochondrial biogenesis, increase thermogenesis (browning) of subcutaneous white adipose tissue, and/or mediate other systemic effects of exercise, in a mammal. In this regard, an exosome dosage sufficient to deliver an amount of one or more metabolic products that increases the plasma level of the one or more metabolic products by at least about 10% of the metabolic product resting plasma level.

In one embodiment, exosomes are isolated according to the isolation protocol described herein. In view of the integrity and stability of the exosomes isolated in this manner, exosome loading of a desired metabolic product in an amount of at least about 1 ng mRNA or miRNA per 10 ug of exosomal protein or 30 ug protein/10 ug of exosomal protein may be achieved.

As will be appreciated by one of skill in the art, prior or subsequent to loading with cargo, the present exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g. peptide having affinity for a receptor or ligand on the target cell or tissue, may be integrated within the exosomal membrane, for example, by fusion to an exosomal membrane marker (as previously described) using methods well-established in the art.

In another embodiment, exersomes may be administered in conjunction with, e.g. in combination with, simultaneously to or separately, at least one additional treatment also effective to increase mitochondrial biogenesis, to enhance or complement the effect thereof. Examples of such additional treatments include, but are not limited to, nutritional or nutraceutical agents (e.g. resveratrol, quercetin, coenzyme Q10, and alpha lipoic acid), massage therapy, exercise (e.g. endurance, resistance or high-intensity interval), medications (e.g. metformin, PPAR agonists, and AICAR), and combinations thereof. In addition, exersomes may be administered in conjunction with a metabolically induced product, as described above, that is administered in a different formulation or by different route of administration.

In another aspect of the invention, an article of manufacture is provided. The article of manufacture comprises packaging material and a composition comprising a pharmaceutically acceptable adjuvant and a therapeutically effective amount of exersomes as defined herein, either isolated from a biological sample as described herein or bio-engineered to incorporate one or more metabolic products. The packaging material is labeled to indicate that the composition is useful to induce mitochondrial biogenesis, increase thermogenesis (browning) of subcutaneous white adipose tissue, and/or mediate other systemic effects of exercise. The packaging material may be any suitable material generally used to package pharmaceutical agents including, for example, glass, plastic, foil and cardboard, and may include instructions for use, including frequency of administration, dosage and the like.

Embodiments of the invention are described by reference to the following example, which is not to be construed as limiting.

### Example 1—Isolation of Exosomes

Blood and urine samples were collected from healthy human subjects. For serum isolation, blood was allowed to clot for 1 hour at room temperature followed by spinning at 2,000×g for 15 min at 4° C. Similarly, urine samples were spun at 2,000×g for 15 min at 4° C. to remove any cellular debris. For plasma isolation, blood was spun down immediately after collection at 2,000×g for 15 min at 4° C. and treated with 5 ug of Proteinase K (20 mg/mL stock, Life Technologies) for 20 min at 37° C. From this point onwards, all samples (serum-1 mL, plasma-1 mL, and urine) are treated exactly the same.

The supernatant from the first centrifugation was spun at 2000×g for 60 min at 4° C. to further remove any contaminating non-adherent cells (optional). The supernatant was then spun at 14,000×g for 60 min at 4° C. (optional). The resultant supernatant was spun at 50,000×g for 60 min at 4° C. The resulting supernatant was then filtered through a 40 μm filter, followed by filtration through a 0.22 μm syringe filter (twice). The filtered supernatant was then carefully transferred into ultracentrifuge tubes and diluted with an equal amount of sterile PBS (pH 7.4, Life Technologies). This mixture was then subjected to ultracentrifugation at 110,000×-170,000×g for 2 hours at 4° C. using a fixed-angle rotor. The resulting pellet was then re-suspended in PBS and re-centrifuged at 110,000×-170,000×g for 2 hours at 4° C. (optional). The pellet was then resuspended carefully with 25 mL of sterile PBS (pH 7.4, Life Technologies) and gently added on top of 4 mL of 30%/70% Percoll™ gradient cushion (made with 0.22 μm filter sterilized water) or 30% Tris/Sucrose/sterile water cushion (300 g protease-free sucrose, 24 g Tris base, 500 ml sterile water, pH 7.4 and 0.22 μm filter sterilized) in an ultracentrifuge tube. This mixture was spun at 150,000×-170,000×g for 90 minutes at 4° C. With a syringe, the exosomal fraction (a distinct pellet at the gradient interface) was isolated carefully, diluted in 50 mL of sterile PBS (pH 7.4, Life Technologies) and spun for 90 minutes at 110,000×-170,000×g at 4° C. to obtain purified exosomes (this is optional when a sucrose gradient is used). The resulting exosomes was resuspended in sterile PBS or sterile 0.9% saline for downstream analyses (in vitro and in vivo). The purity of the exosomal fraction was confirmed by sizing, immuno-gold labelling/Western blotting using at least two independent exosome membrane markers, in this case, CD9 and CD63 were used.

The protocol was also used to isolate exosomes from 1 mL of serum obtained from C57B1/6J mice, and from conditioned media from human and mouse immature dendritic cell culture. Immature dendritic cells from human and mice are grown to 65-70% confluency in alpha minimum essential medium supplemented with ribonucleosides, deoxyribonucleosides, 4 mM L-glutamine, 1 mM sodium pyruvate, 5 ng/mL murine GM-CSF, and 20% fetal bovine serum. For conditioned media collection, cells were washed twice with sterile PBS (pH 7.4, Life Technologies) and the aforementioned media (with exosome-depleted fetal bovine serum) was added, and conditioned media was collected after 48 hours.

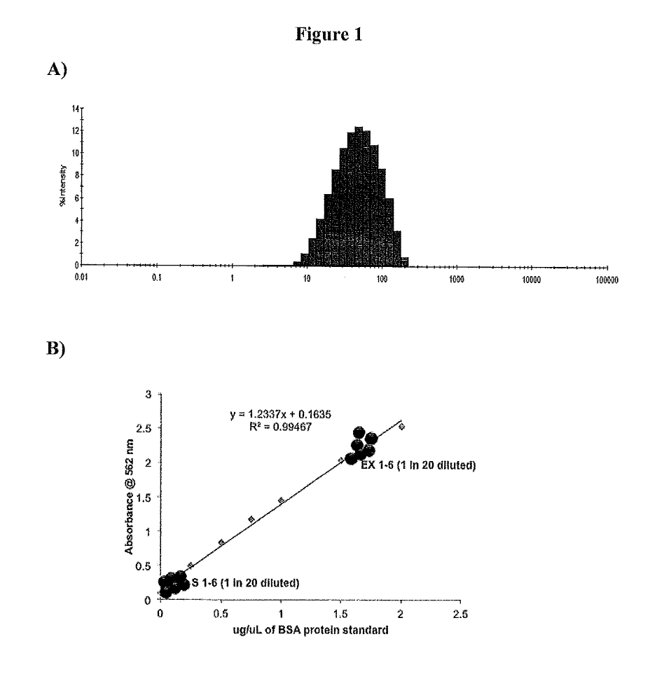

A BCA assay (Pierce™) was used to determine the yield of exosomes in each sample. The yield from serum, plasma and urine was determined to be in the range of 2-20 μg/μL, while the purity of the exosomal fraction was confirmed by qualitative immunogold-labelling, which indicated an average particle diameter of 90 nm, with minimal contamination outside of the 20-120 nm size range (FIG. 1A). The stability of the exosomes was also determined using a Beckman DelsaMax dynamic light scattering analyzer. The zeta potential of exosomes isolated from serum was determined to be −80.4 mV.

This isolation protocol was compared to commercially available exosome isolation kits from Life Technologies™, Cell Guidance System™, Norgen Biotek™ Corporation, Qiagen™, Exigon™, and System Biosciences™ according to manufacturer's instructions. The quality of exosomes isolated using these kits was quite inferior to the quality of exosomes isolated as described above. Specifically, as determined by electron microscopy analyses, the commercial kits yield a product containing contaminating debris and clumped microvesicles, while the above protocol yielded circular exosomes having an average diameter of 90 nm that were not clumped. The quantity of exosomes isolated using the above protocol was notably greater (10-25 μg/μL total protein as determined by BCA protein assay) than the protein quantity isolated using any of the commercial kits tested (0.1-0.5 μg/μL total protein as determined by BCA protein assay). Thus, the current protocol yielded about 80-100×more exosomes (EX1-EX6) in comparison to the protein yield of commercially available kits (S1-S6) as illustrated in FIG. 1B, In addition, the products isolated using commercial kits exhibited poor stability having a zeta potential of greater than −10 mV (i.e. between −10 to 0 mV), and exhibited rapid coagulation/flocculation, in stark contrast to the stability of the exosomes isolated by the above protocol. It was also noted that the products isolated using commercial kits were quite insoluble in physiological buffers as compared to the solubility in physiological buffer of the exosomes isolated using the present methods of Examples 1-3. The pellet obtained using commercially available kits could not be efficiently suspended in physiological buffer or in detergent-based buffers such as RIPA buffer or urea buffer.

### Example 2—Exosome Isolation Using PEG-Based Method

Exosomes were isolated from various human and other mammalian biological samples as follows.

Blood samples were collected from healthy human subjects using red top serum collection tubes (e.g. BD, Ref #367812) and blue top plasma collection tubes containing sodium citrate (e.g. BD, Ref 14369714) for serum and plasma isolations, respectively. For serum isolation, blood was allowed to clot for 1 hour at room temperature followed by centrifugation at 2,000×g for 15 min at 4° C. For plasma isolation, blood was spun down immediately after collection at 2,000×g for 15 min at 4° C. Plasma and serum was similarly collected from C57B1/6J mice and Sprague Dawley rats. Exosomes were then isolated from these samples, as well as from bovine whole milk (Natrel fine-filtered 3.25% milk) and cells in culture (e.g. CHO cells). From this point onwards, all exosome sources were treated the same.

Serum, plasma and milk were spun at 2000×g for 15 min at 4° C. The supernatant from the first centrifugation was spun at 2000×g for 60 min at 4° C. to pellet debris. The supernatant was then spun at 15,000×g for 60 min at 4° C. The resulting supernatant was then filtered through a 45 μm filter (Millipore, cat. # SLHV033RS), followed by filtration through a 0.22 μm syringe filter (Millipore, cat. # SLGP0334B). The centrifugation and filtering steps have been determined to be optional steps. The filtered supernatant was then added to an equal volume of 16% PEG 6000 (Sigma, cat. #81253) and 500 mM NaCl in PBS (Bioshop, cat. # SOD002), mixed by inversion or gentle pipetting and incubated for 30 min at 4° C. The filtrate-PEG (8%) solution was then spun at 10,000×g for 10 min at 4° C. to pellet the exosomes. The supernatant was discarded and the pellet was solubilized in 600 uL of 0.5M trehalose (Sigma, cat. # T0167) in PBS by gentle pipetting or on a mechanical plate rocker for 30 min at 4° C. Exosomes were further purified by applying the exosome-containing solution to centrifugation between 15,000×g-150,000×g for 1 hr at 4° C. The resulting supernatant containing purified exosomes was then collected.

A BCA assay (Pierce™) was used to determine exosome yield of between 5-10 mg of exosomal protein per 1 mL of serum used. Transmission electron microscopy was performed on exosome solutions confirming the isolation of exosomes in the size range of 20-140 inn in diameter. The size distribution profile of exosomes isolated using the present PEG-based method was then measured using a Beckman DelsaMax dynamic light scattering analyzer, showing that the majority of particles in these solutions were within the 20-140 nm size range with minimal contamination outside of this exosome size range. Exosomal purity was further exemplified by performing Western blots with the canonical exosome markers CD9, CD63, CD81 and TSG101. Both the supernatant and pellet fractions of exosome solutions isolated from mouse serum and plasma samples using the PEG-based isolation method (and a final ultracentrifugation step) demonstrated robust expression of these markers confirming the presence of exosomes. The purity of exosomes was also determined by performing a Ponceau S stain, a widely used indicator for the presence of protein bands during Western blotting.

### Example 3—Effect of Exercise on Exosomes

Mice were divided into sedentary (SED) or acute endurance exercise groups (END; 15 min or 30 min or 90 min, 15 m/min) group. Serum was obtained from each group, and immediately following an acute bout of exercise for END groups. Exosomes were isolated from 1 mL of serum obtained from C57B1/6J mice using the method as described in Example 1, Nanoparticle tracking analyses and sizing analyses of isolated exosomes from serum of mice in SED and END groups were conducted.

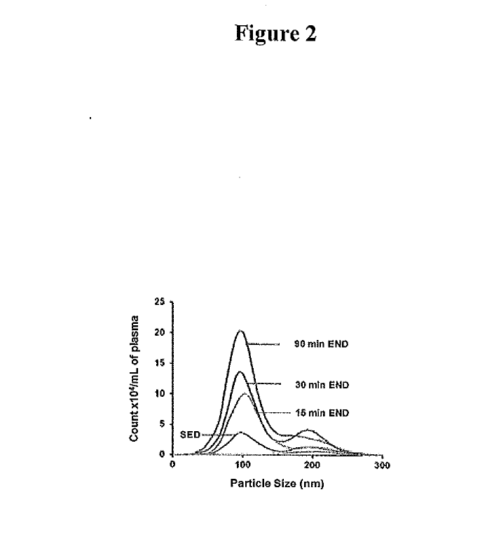

Serum exosomal content was found to increase with increasing duration of acute endurance exercise as shown in FIG. 2. Exosomes isolated from mouse serum were determined to have an average size of about 90 nm.

### Example 4—Effect of Exosomes on Dermal Fibroblasts

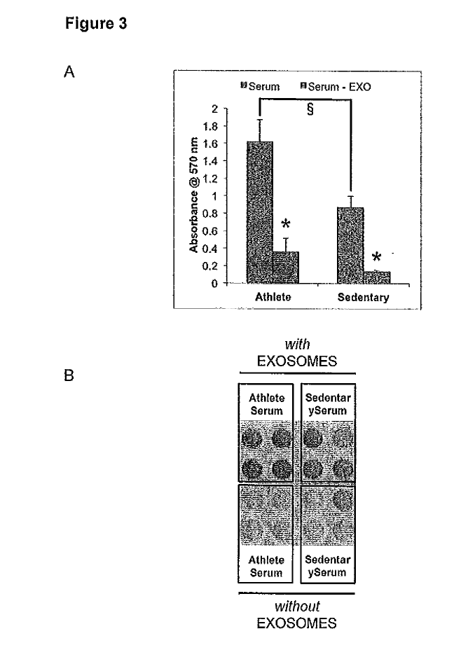

Human dermal fibroblasts were treated with either serum or exosome-depleted serum (Serum-EXO) from athletes and sedentary men for 5 days in culture (n=3/treatment). A cell proliferation assay (Vybrant® MTT) was conducted on the treated fibroblasts to identify cellular proliferation/viability. Data were analyzed using an unpaired t-test and are presented as mean±SEM.*P<0.05 for Serum vs. Serum-EXO groups; §P<0.05 for Athlete vs. Sedentary Serum groups. As shown in FIG. 3, fibroblast proliferation was greatest in fibroblasts treated with exosome-containing serum from athletes as opposed to serum from sedentary men, while proliferation was severely attenuated in fibroblasts treated with exosome-depleted serum from athletes or sedentary men.

Blood draws were obtained prior to and immediately following an acute bout of endurance exercise (65% of VO₂ₘₐₓ, 60 min on cycle ergometer). Human dermal fibroblasts were treated with total serum obtained either pre- or post- an acute bout of endurance exercise, or with isolated exosomes from pre- or post-exercise serum, or with exosome-depleted serum (EDS) from pre- or post-exercise serum, for 5 days in culture (n=3/treatment). Vybrant® MTT cell proliferation assay was carried out to identify cellular proliferation/viability. Data were analyzed using an unpaired t-test and are presented as mean±SEM.*P<0.05 from all other groups.

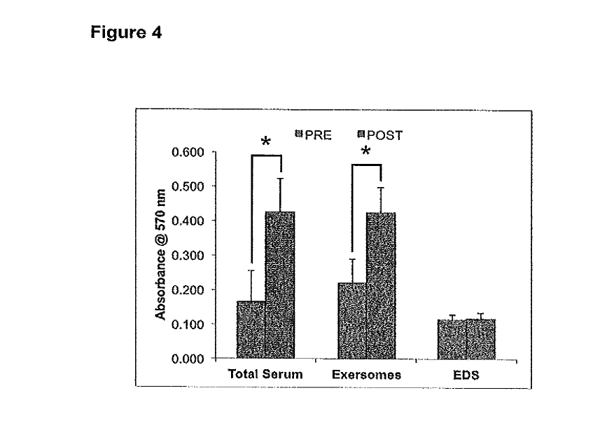

As shown in FIG. 4, post-exercise total serum and exosomes exhibit significant fibroblast proliferative capacity, while exosome-depleted serum prepared from serum isolated following an acute bout of exercise loses its capacity to maintain and rejuvenate human dermal fibroblasts. Thus, exersomes contain proliferative components, i.e. exerkines. Trypsinization/heat inactivation/RNase treatment of exercise serum abolishes its pro-metabolic and proliferative activity. This indicates that the exersome-encapsulated exerkines are primarily peptides, mRNA and miRNA species.

### Example 5—Effect of Exosome Administration

Since exosomes appear to incorporate exerkines that impart the systemic effects of endurance exercise, the effects of infusion of exosomes alone was determined.

Exosomes were isolated from serum obtained from sedentary (SED) or endurance exercise trained (END; treadmill training: 15 m/min for 60 min, 5×/week for 2 months) C57B1/6J mice using modified-ultracentrifugation methodology. Isolated SED and END exosomes were reconstituted in sterile saline and were injected intravenously to an independent cohort of sedentary C57Bl6J mice (5×/week with 1 to 1 donor-recipient ratio—exosomes isolated from approximately 200 μl of mouse serum). After 6 weeks of treatment, sedentary mice getting (A) SED exosomes or (B) END exosomes (exersomes) were transferred to voluntary wheel running cages for three days to measure their basal voluntary endurance activity in day-night (white-black bars) cycle.

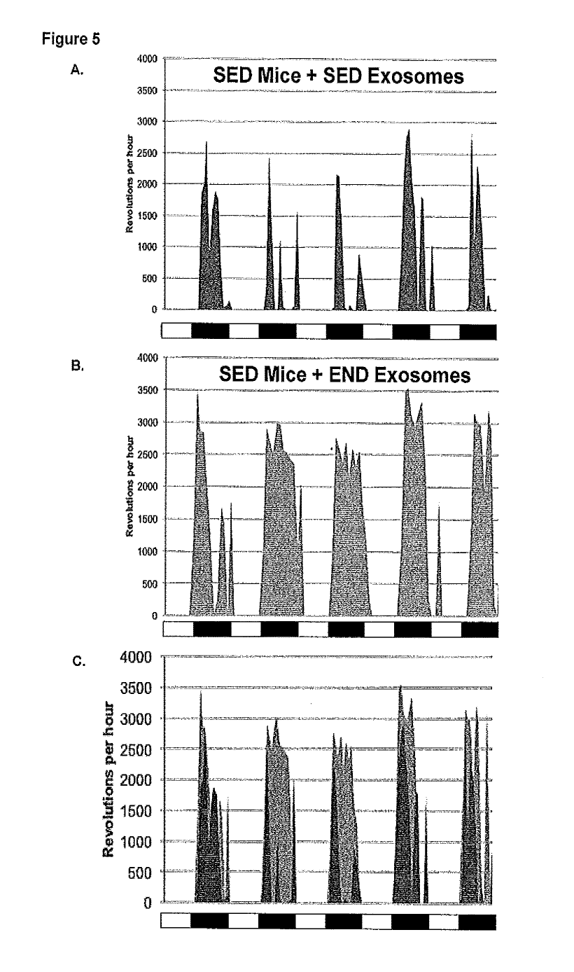

An increase in basal voluntary activity of sedentary mice was observed when given intravenous boluses of exosomes from exercised-mice (END) vs. exosomes from sedentary mice (SED) (see FIG. 5A/B).

In a similar study, Isolated END exosomes were reconstituted in sterile saline and were injected intravenously into an independent cohort of sedentary C57B1/6J mice (5×/week with 1 to 1 donor-recipient ratio). A separate cohort of C57B1/6J mice were trained using voluntary wheel running cages for 10 weeks. After 10 weeks of treatment, basal voluntary activity of endurance-trained mice and sedentary mice receiving exersomes (END exosomes) were transferred to voluntary wheel running cages for three days to measure their basal endurance voluntary activity in day-night (white-black bars) cycle.

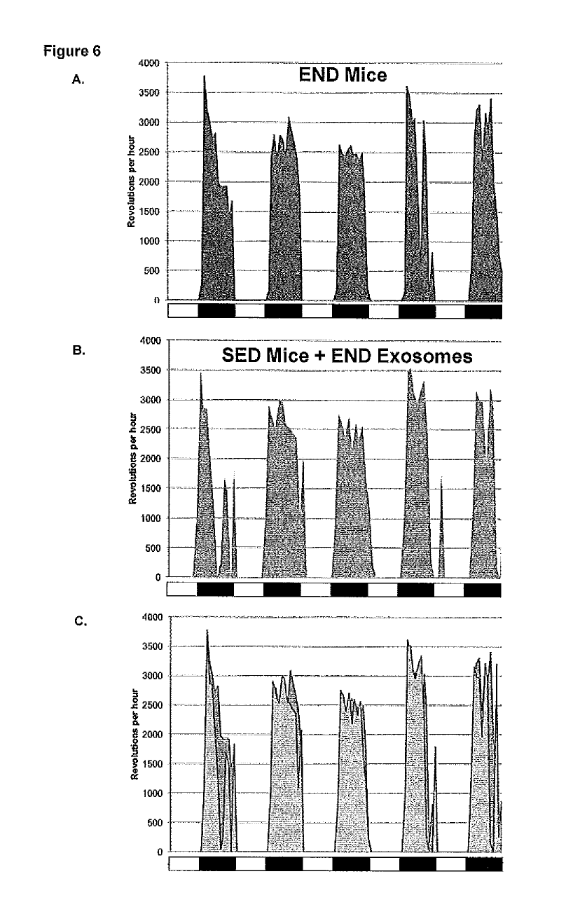

The basal voluntary activity of sedentary mice that received END exersomes for 10 weeks while maintaining their ‘sedentary status’ (i.e., wheel cage was locked to prevent exercising) (FIG. 6B) was comparable to that of mice that were trained in voluntary wheel cages for 10 weeks (FIG. 6A). An overlay of the voluntary wheel activity of both groups is shown in FIG. 6C and illustrates the similar profiles of each group.

Isolated SED and END exosomes as above were injected intravenously to an independent cohort of sedentary C57B1/6J mice (5×/week with 1 to 1 donor-recipient ratio). After 6 weeks of treatment, sedentary mice getting SED exosomes or END exosomes (exersomes) were subjected to a treadmill-based endurance stress test to exhaustion. Additionally, a separate cohort of C57B1/6J sedentary (SED) or endurance trained mice (END; trained in voluntary wheel running cages for 10 weeks) were subjected to endurance stress test as negative and positive control of endurance exercise adaptations, respectively. Data were analyzed using an unpaired t-test and are presented as mean±SEM.*P<0.05 for SED vs. END groups; §P<0.05 for SED+SED EXO vs. SED+END EXO groups.

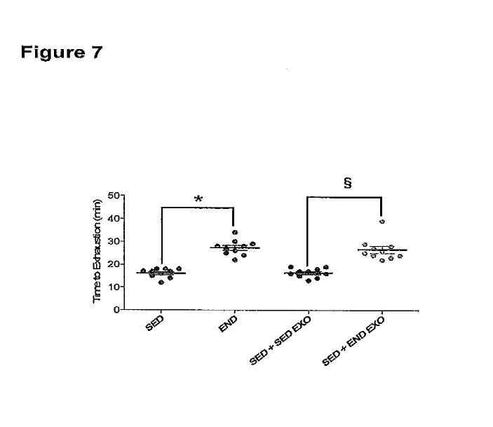

As shown in FIG. 7, SED mice treated with END exosomes exhibited similar endurance to END mice, while SED mice treated with SED exosomes did not show any change in endurance.

### Example 6—Effect of Exersomes Against Diet-Induced Obesity/Type 2 Diabetes

Since exosomes alone can increase exercise tolerance in mice naïve to any endurance exercise training, it was determined whether or not exosomes alone can therapeutically reverse high-fat diet induced obesity and type 2 diabetes.

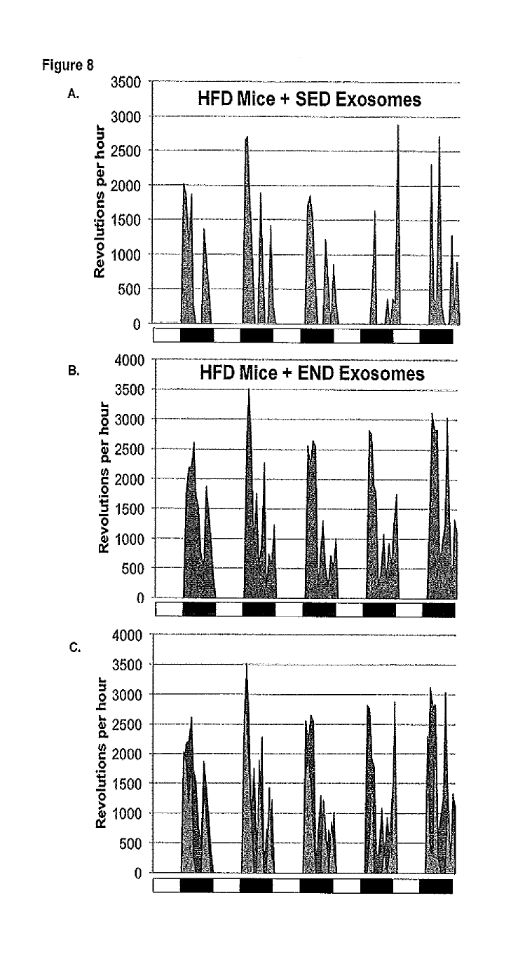

SED and END exosomes obtained as in Example 4 and were injected intravenously to an independent cohort of high-fat diet (HFD; 45% kCal from fat) C57B1/6J mouse model of obesity (1 to 1 donor-recipient ratio). After 10 weeks of treatment, HFD fed mice were transferred to voluntary wheel running cages for three days to measure their basal voluntary endurance activity in day-night (white-black bars) cycle. As shown in FIG. 8, HFD mice treated with END exosomes exhibited increased basal voluntary endurance activity (FIG. 8B) as compared to HFD mice treated with SED exosomes (FIG. 8A). Differences in voluntary wheel activity of both groups are shown in an overlay of the results (FIG. 8C).

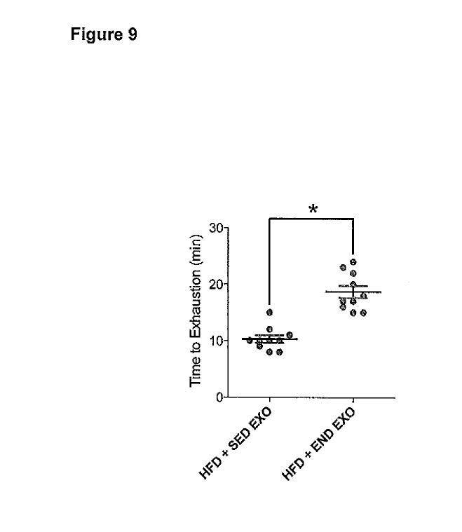

A similar experiment to the above was conducted; however, after 10 weeks of treatment, HFD fed mice receiving SED exosomes or END exosomes (exersomes) were subjected to a treadmill-based endurance stress test to exhaustion. Data were analyzed using an unpaired West and are presented as mean±SEM.*P<0.05 for HFD+SED EXO vs. HFD+END EXO groups. An increase in maximum endurance capacity (FIG. 9) of high-fat diet fed (HFD) mice receiving END exosomes was observed versus HFD mice receiving SED exosomes from sedentary mice.

After 10 weeks of treatment, HFD fed mice getting SED exosomes or END exosomes (exersomes) were (A) weighed and (B) subjected to a glucose tolerance test (GTT).

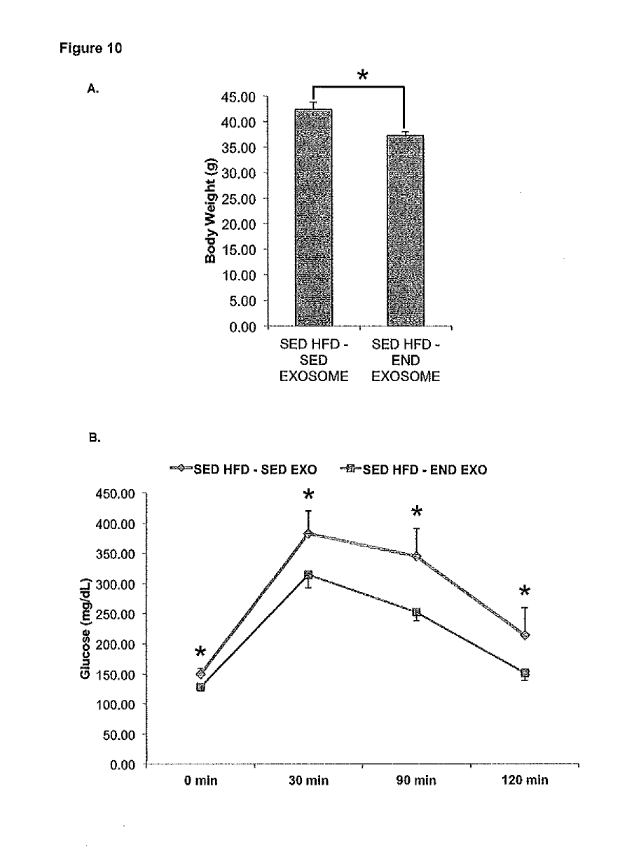

In addition, following 10 weeks of treatment with SED or END exosomes, the HFD mice were weighed and subjected to a glucose tolerance test (GTT). Data were analyzed using an unpaired t-test for weight and one-way ANOVA for OTT, and are presented as mean±SEM.*P<0.05 for all groups. The treatment with END exosomes resulted in a significant reduction in body weight of HFD mice (see FIG. 10A), and improved glucose tolerance/metabolism (FIG. 10B).

### Example 7—Effect of Exersomes on Mitochondrial Disease and Aging

Eight month old POLG1 mutator mice show early signs of aging, display vast array of systemic pathologies, are exercise intolerant and possess a primary genetic mitochondrial mutation that results in systemic mitochondrial dysfunction, and multisystem pathology.

### Breeding of PolG Mutator Mouse and Littermate Wildtype Mice

Heterozygous mice (C57B1/6J, PolgA+/D257A) for the mitochondrial polymerase gamma knock-in mutation were a kind gift of Dr. Tomas A. Prolla, University of Wisconsin-Madison, USA (as described in Kujoth. Science 309, 481-484 (2005)). Homozygous knock-in mtDNA mutator mice (PolG; PolgAD257A/D257A) and littermate wildtype (WT; PolgA+/+) were generated from heterozygous mice-derived colony maintained at the McMaster University Central Animal Facility as previously described (Safdar et al. Proc Natl Acad Sci USA 108, 4135-4140 (2011)). During breeding, all animals were housed three to five per cage in a 12-h light/dark cycle and were fed ad libitum (Harlan-Teklad 8640 22/5 rodent diet) after weaning. The presence of the polymerase gamma homozygous knock-in mutation was confirmed as previously described (Safdar et al. Proc Nal Acad Sci USA 108, 4135-4140 (2011)).

### Endurance Exercise Protocol for PolG Mutator Mice

Endurance exercise protocol and tissue harvesting was carried out as previously described using an independent cohort of mice (Safdar et al. Proc Natl Acad Sci USA 108, 4135-4140 (2011)). Briefly, at three months of age, mice were housed individually in micro-isolator cages in a temperature- and humidity-controlled room and maintained on a 12-h light-dark cycle with food and water ad libitum. Equal numbers of PolG female and male mice were assigned to sedentary (PolG-SED) or forced-endurance (PolG-END) exercise groups (n=10/group; ♀=♂). None of the mice had been previously subjected to a structured exercise regimen. One week of pre-training was allowed to acclimatize the PolG-END mice to the treadmill. PolG-END mice were subjected to forced treadmill exercise (Eco 3/6 treadmill; Columbus Instruments, Columbus, Ohio) three times per week at 15 m/min for 45 min for five months. A 5-min warm-up and cool-down at 8 m/min was also included. PolG mice were age- and sex-matched with sedentary littermate WT mice (n=10; ♀=♂), which served as controls for the study to assess if endurance exercise intervention can molecularly bring PolG mice to normalcy. At eight months of age animals were euthanized, and tissues were collected for molecular analyses. The study was approved by the McMaster University Animal Research and Ethics Board under the global Animal Utilization Protocol #12-03-09, and the experimental protocol strictly followed guidelines put forth by Canadian Council of Animal Care. At the end of the study, pancreas was harvested and stained with anti-insulin antibody (Abcam).

### Administration of Exosomes

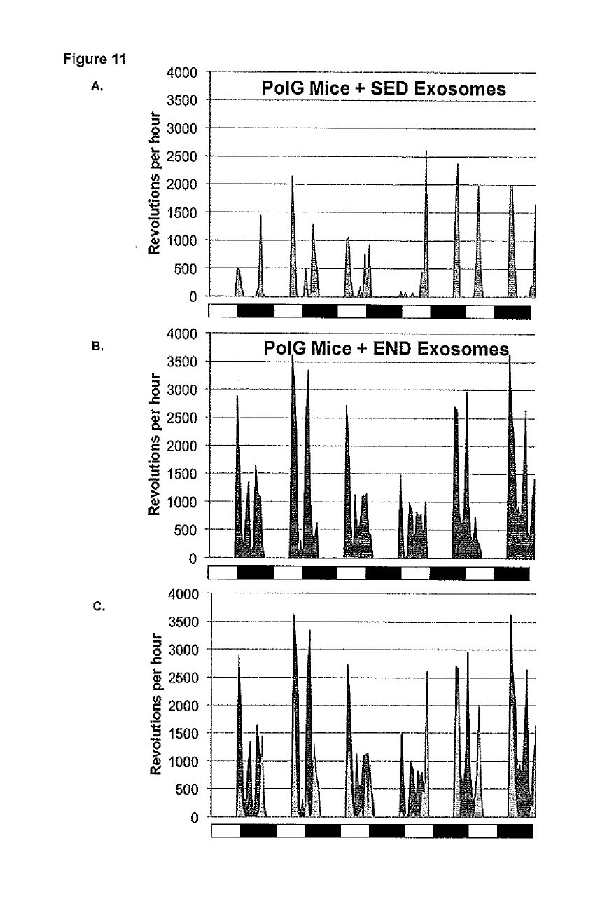

Isolated SED and END exosomes, as described in Example 4, were injected intravenously to an independent cohorts of PoLG mice, SED and END (8 months old; 1 to 1 donor-recipient ratio). After 8 weeks of treatment, mice were transferred to voluntary wheel running cages for three days to measure their basal voluntary endurance activity in day-night (white-black bars) cycle. As shown in FIG. 11, PoLG mice treated with END exosomes exhibited increased basal voluntary endurance activity (FIG. 11B) as compared to PoLG mice treated with SED exosomes (FIG. 11A). Differences in voluntary wheel activity of both groups are shown in an overlay of the results (FIG. 11C).

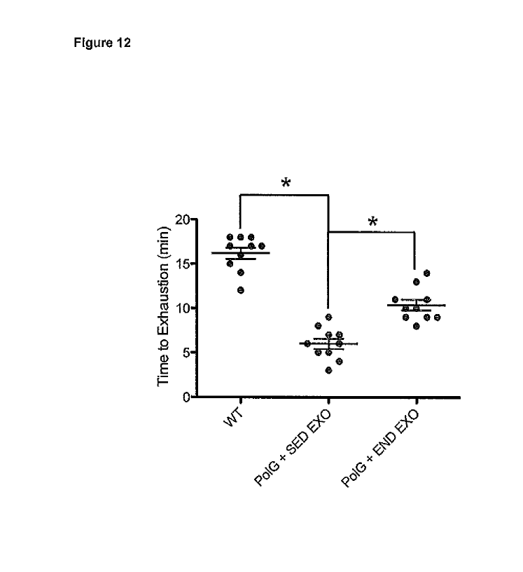

In a similar experiment, after 8 weeks of treatment, treated PoLG mice were subjected to a treadmill-based endurance stress test to exhaustion. PoLG mice treated with END exosomes exhibited increased maximum endurance capacity as compared to that of SED exosome treated mice as shown in FIG. 12. Data were analyzed using an unpaired t-test and are presented as mean±SEM.*P<0.05 for all groups.

### Example 8—Effect of Exersomes on Mitochondrial Biogenesis

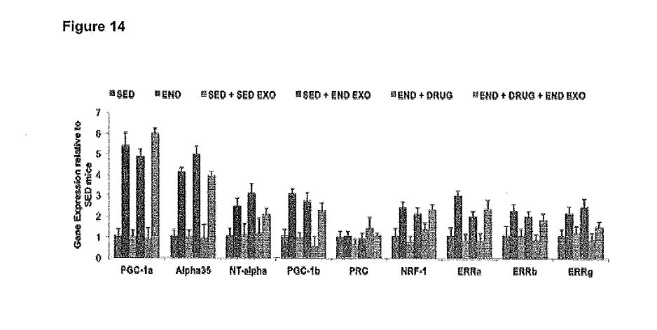

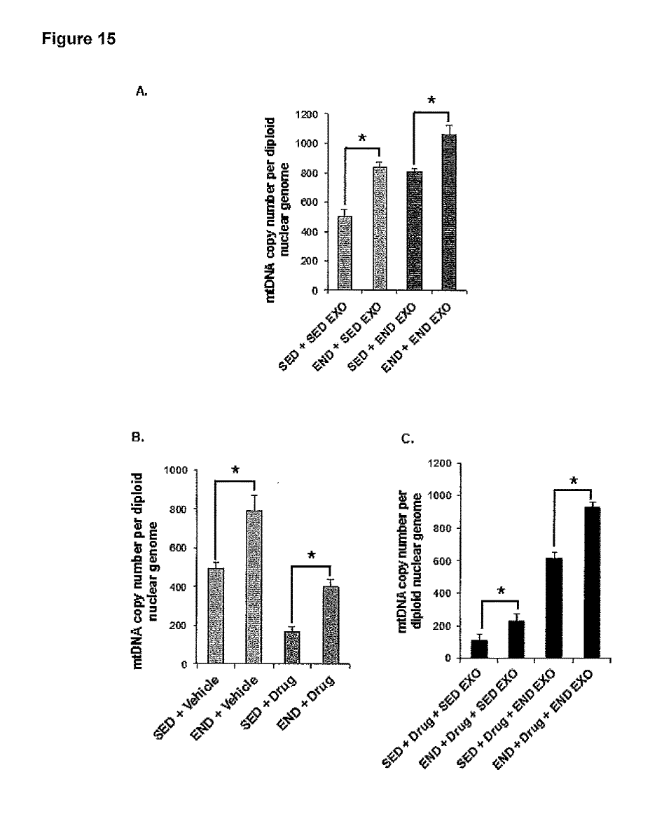

Exosomes were isolated from serum obtained from sedentary (SED) or endurance exercise trained (END; treadmill training: 15 m/min for 60 min, 5×/week for 3 months) C57B/6J mice using ultracentrifugation methodology (as described in Example 1). Isolated SED and END exosomes were reconstituted in sterile saline and were injected intravenously to an independent cohort of sedentary C57B1/6J mice (5×/week with 1 to 1 donor-recipient ratio (exersomes isolated from approximately 200 μl)). Additionally, there are two separate groups of mice that were exercised trained as described (using the aforementioned protocol) and were injected intraperitoneally with exosome secretion inhibitor, GW4869 (1 ug/g of mouse in 0.9% sterile saline, 5×/week). One of these groups (END+Drug+END-EXO) was then injected with END exosomes (5×/week with 1 to 1 donor-recipient ratio). After 12-weeks of treatment, skeletal muscle of sedentary mice getting END exosomes (EXERSOMES) showed a significant increase in PGC-1α-mediated mitochondrial biogenesis gene signature comparable to mice that were exercised (FIG. 14). Exosome inhibitor (GW4869) prevented endurance exercise-mediated increase in mitochondrial biogenesis (FIG. 14). Similarly, endurance exercise-mediated increase in mtDNA copy number was recapitulated in muscle harvested from sedentary mice that were infused with END exosomes (EXERSOMES), while GW4869 prevented this increase (FIG. 15 A-C).

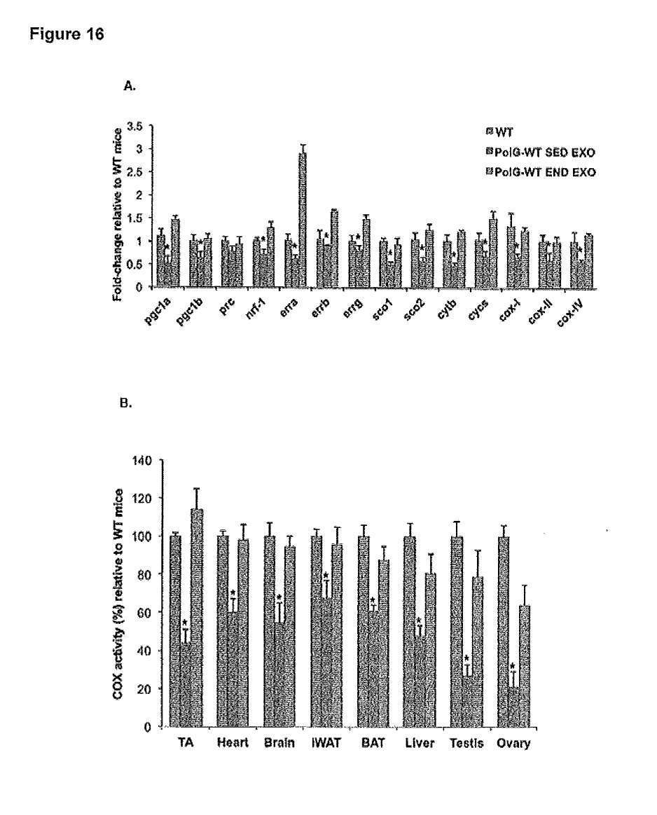

Mitochondrial DNA mutator mice (PolG) possess a knock-in mutation in the proof-reading domain of mitochondrial polymerase gamma. This results in accelerated aging, many aspects of which phenocopy human aging, including: sarcopenia, cardiomyopathy, brain atrophy, gonadal atrophy, osteoporosis, kyphosis, etc. Treatment of PolG mice with exosomes from WT-SED mice or WT-END mice (5×/week with 1 to 1 donor-recipient ratio for 12 weeks) resulted in induction of PGC-1α-mediated mitochondrial biogenesis gene signature (FIG. 16A) and a systemic increase in mitochondrial cytochrome c oxidase activity (FIG. 16B).

### Example 9—Effect of Exersomes on Beige Fat Gene Expression

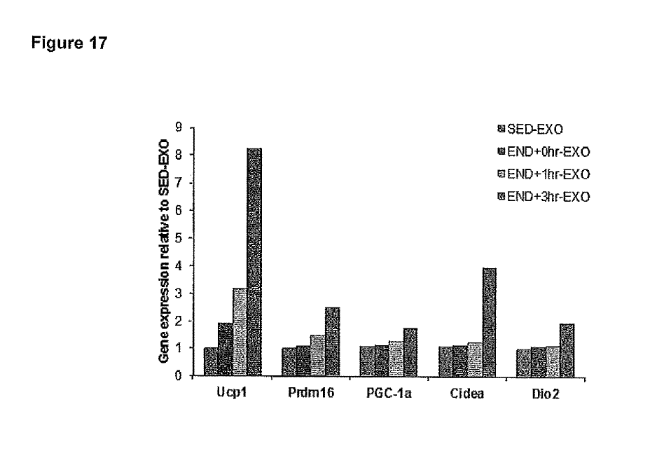

C57B1/6J mice were subjected to an acute treadmill run (15 m/min for 90 min), and were harvested immediately after, 1 hour after, or 3 hours after exercise. A group of sedentary mice served as the control. Exersomes were isolated as described in Example 1 from the serum of the exercised and control mice. The isolated exersomes were then administered (100 ug of total exersomal protein reconstituted in 200 uL of sterile saline) to primary human subcutaneous pre-adipocytes (cell line purchased from ATCC, Cat. #PCS-210-010) during 5 days of differentiation. Exersomes from mice subjected to acute endurance exercise exhibited induced beige fat gene expression, namely expression of Ucp1, Prdm16, PGC-1α, Cidea and Dio2 (Type II iodothyronine deiodinase gene), in primary human subcutaneous pre-adipocytes (FIG. 17).

### Example 10—Proteomics and RNA-Sea Analyses of Exersomes

A proteomics screen (using standard Mass Spectrometry—LC/MS/MS platform) and genomics screen (using standard Illumina® RNA-Seq platform) was run on exersomes obtained as described previously to determine protein and gene content. FIGS. 18A and 18B illustrate the results of the protein and gene screens, respectively. miRNA is identified by reference to NCBI (National Centre for Biotechnology Information) reference number. Some highly expressed proteins in the exersomes were determined as shown in Table 1 below, and miRNA content (present in an amount at least 10-fold greater than that found in exosomes isolated from non-exercised mammals) is shown in FIG. 18B.

TABLE 1

| Protein | Protein Accession Number |
|---|---|
| PDGFB | EDL04617 |
| METRNL | AAH24497 |
| FNDC5 | AAI09185 |
| FNDC4 | AAH27164 |
| SHISA5 | AAH17600 |
| SPP1 | CAJ18565 |
| PIP | AAA75283 |
| TPM1 | AAI45308 |
| PSAP | NP_075623 |
| VEGF-B | AAH46303 |

### Example 11—Activity of METRNL

To determine if one or more of the highly expressed proteins in exersomes exhibits the effects of the exersomes (as described in the foregoing Examples), the effect of the protein, METRNL, was studied.

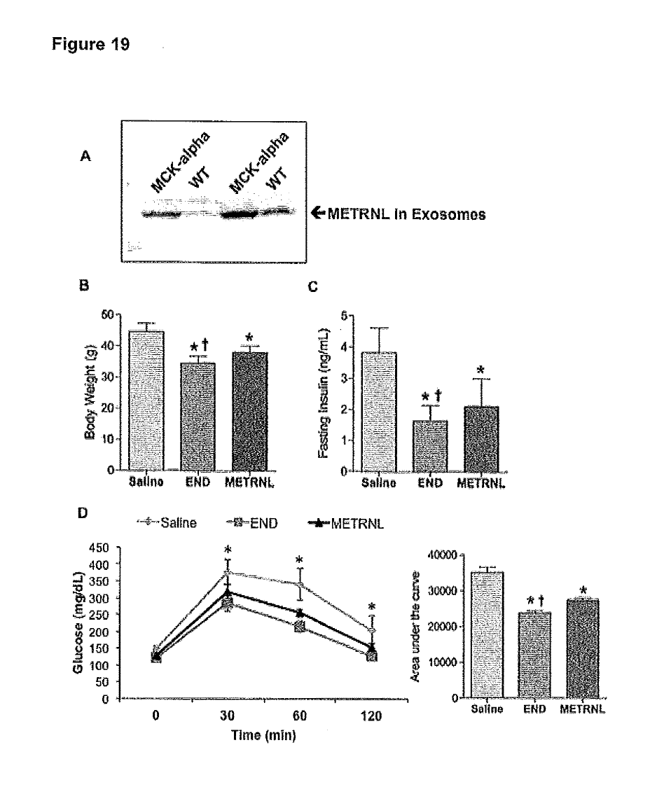

First, it was confirmed that METRNL was present in exosomes using Western blotting. Exosomes were isolated from MCK-PGC-1a mice (obtained from Jackson Laboratories) and littermate wildtype controls, for example, as described in Example 1. Since METRNL is regulated by muscle PGC-1a, exosomes from MCK-PGC-1a mice showed increased METRNL protein content vs. exosomes from littermate wildtype mice (FIG. 19A).

Next, C57BL/6 mice fed a 60% kcal high-fat diet for 20 weeks were injected with saline (control group), or subjected to forced-treadmill running (END exercise group, 15 m/min for 60 min; 5×week for 2 months), or injected with recombinant mouse METRNL (experimental group; 0.4 ng/kg in 150 uL of 0.9% sterile saline) intravenously for 4 weeks (n=8 per group). Mice injected with METRNL showed a significant reduction in their body weight (FIG. 19B), improved fasting insulin (FIG. 19C) and improved glucose tolerance (FIG. 19D).

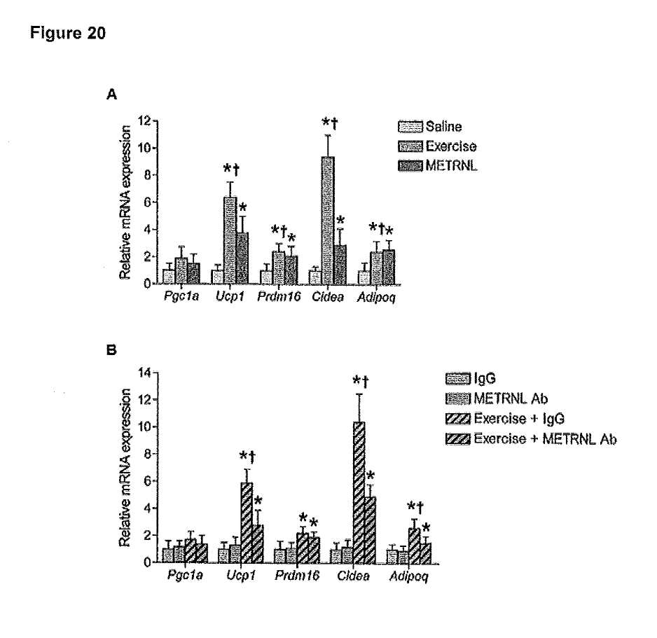

Furthermore, the effect of METRNL on beige fat gene expression in primary human subcutaneous pre-adipocytes was determined as described in Example 8. It was determined that beige fat gene expression was induced, at least in part, by METRNL (FIGS. 20 A-B).

### Example 12—Activity of FNDC5

To determine if another of the highly expressed proteins in exersomes exhibits the effects of the exersomes (as described in the foregoing Examples), the effect of FNDC5 (fibronectin type III domain containing 5) protein was determined.

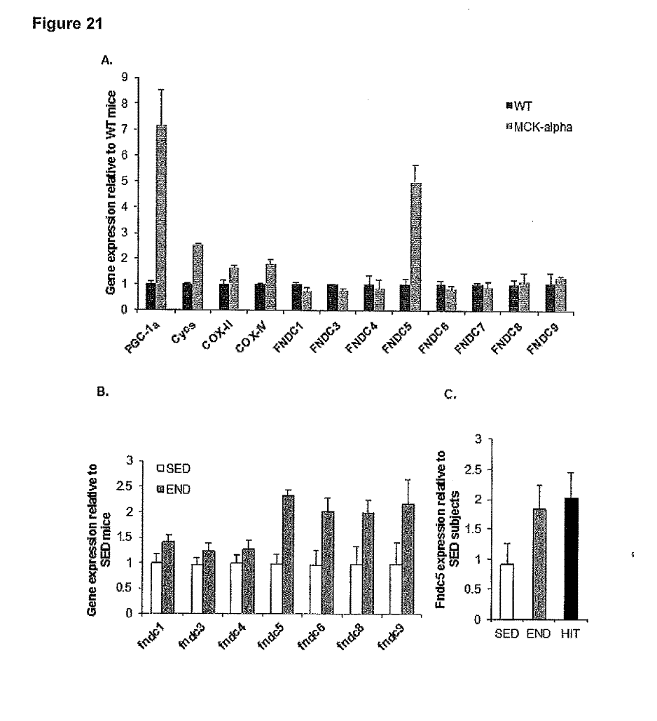

It was first determined that the fndc5 gene was induced in response to transgenic over-expression of PGC-1a in muscle (FIG. 21A), and was also induced in skeletal muscle in response to endurance exercise training in mice (FIG. 21B) and in humans (FIG. 21C—endurance exercise (END), high intensity interval training (HIT)).

The effect of exercise training on FNDC5 protein content in humans. Western blot analyses (using Phoenix Pharmaceutical Antibody #0-067-17) was conducted in human skeletal muscle biospy homogenates, and a specific band (˜28 kDa) was seen confirming that FNDC5 protein content is exercise responsive. The same antibody was used to assess the presence of FNDC5 in exosomes isolated from mice (sedentary vs. endurance-trained). An increase in exosomal FNDC5 content was found in response to exercise training in mice. The presence of the proteolytic by-product of FNDC5, irisin (˜12 kDa peptide), was not detected.

It was then determined if the FNDC5 that is produced in skeletal muscle is packaged in exosomes before it is released into the circulation. An immunohistochemistry analysis of FNDC5 (using Phoenix Pharmaceutical Antibody #G-067-19) localization in muscle in relation to exosomes (ALIX used as a marker of exosomes) confirmed that, once produced by the muscle, FNDC5 is packaged into exosomes before being released systemically. Furthermore, to confirm that the band observed in serum exosomes is FNDC5 and not irisin, protein de-glycosylation treatment was conducted. Treatment of exosomal homogenate with a Protein Deglycosylation Mix (New England Biolabs, which contains PNGase F, O-glycosidase, neuraminidase, β1-4-glalactosidase, and 1-N-acetylglucosaminidase) does not result in reduction of molecular weight, confirming the presence of FNDC5 and not irisin.

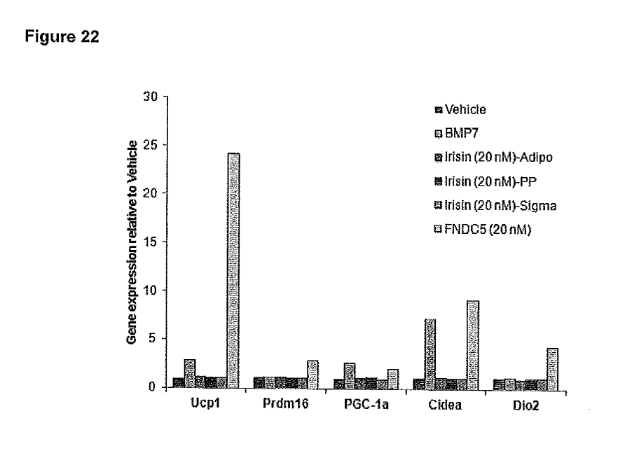

The activity of FNDC5 and irisin to induce browning of subcutaneous fat was then determined. Recombinant irisin from three credible sources (Adipogen, Phoenix Pharmaceuticals, and Sigma) and recombinant FNDC5 protein, variant 4 (Phoenix Pharmaceutical #067-18) were each administered to and incubated with primary human subcutaneous pre-adipocytes during 5 days of differentiation. BMP7, a potent inducer of fat browning was used as a positive control. While FNDC5 and BMP7 were shown to induce the browning gene expression program in pre-adipocytes, irisin did not (FIG. 22). Thus, FNDC5 exhibits a functional capacity with respect to browning of white adipose tissue.

### Details of Methods Used in the Examples

### Acute Endurance Exercise Protocol for Wildtype Mice

Male C57B1/6J mice, bred in an institutional central animal facility (McMaster University), were housed in micro-isolator cages in a temperature- and humidity-controlled room and maintained on a 12-h light-dark cycle with food and water ad libitum. At 4 months of age, mice (N=10/group) were randomly assigned to either sedentary (SED) or forced-acute endurance exercise post 1-hour (Acute END+1 hr) or forced-acute endurance exercise post 3-hour (Acute END+3 hr) groups ensuring that body mass was similar between groups. None of the mice had been previously subjected to a structured exercise regime. Mice in both exercise groups were subjected to an acute bout of treadmill (Eco 3/6 treadmill; Columbus Instruments, Columbus, Ohio) running at 15 m/min for 90 min. A 5-min warm-up and cool-down at 8 m/min was also included. All of the mice in END exercise group completed the 90 min trial and were visibly exhausted (i.e., mouse will sit at the lower end of the treadmill, on the shock bar, for 0.5 seconds). Mice in the SED group served as controls. One or three hours following the acute bout of exercise, mice liver, heart, fat pads (inguinal and brown adipose tissue), and skeletal muscle (quadriceps) were harvested. Our exercise studies Animal Utilization Protocol is approved by the McMaster University Animal Research and Ethics Board under the global Animal Utilization Protocol #12-03-09, and the experimental protocol strictly followed guidelines put forth by Canadian Council of Animal Care.

Total RNA Isolation and mRNA Expression Analyses

Total RNA was isolated from tissues (liver, heart, fat, and skeletal muscle) using the Qiagen total RNA isolation kit (Qiagen, Mississauga, ON.) according to the manufacturer's instructions. RNA samples were treated with TURBO DNA-freeTM (Ambion Inc., Austin, Tex.) to remove DNA contamination. RNA integrity and concentration was assessed using the Agilent 2100 Bioanalyzer (Agilent Technologies, Palo Alto, Calif.). The average RIN (RNA integrity number) value for all samples was 9.2±0.2 (scale 1-10), ensuring a high quality of isolated RNA. The mRNA expression of genes involved in metabolism and browning gene program were quantified using 7300 Real-time PCR System (Applied Biosystems Inc., Foster City, Calif.) and SYBR® Green chemistry (PerfeCTa SYBR® Green Supermix, ROX, Quanta BioSciences, Gaithersburg, Md.) as previously described 66. First-strand cDNA synthesis from 500 ng of total RNA was performed with random primers using a high capacity cDNA reverse transcription kit (Applied Biosystems Inc., Foster City, Calif.) according to manufacturer's directions. Forward and reverse primers for the aforementioned genes were designed based on sequences available in GenBank using the online MIT Primer 3 designer software (developed at Whitehead Institute and Howard Hughes Medical Institute by Steve Rozen and Helen Skaletsky), and were confirmed for specificity using the basic local alignment search tool. β-2 microglobulin was used as a control house-keeping gene, as its expression was not affected with the experimental intervention. All samples were run in duplicate simultaneously with a negative control which contained no cDNA.

### Total DNA Isolation

Total DNA (genomic and mtDNA) I was isolated from tissue or cells using the QIAamp DNA Mini kit (Qiagen, Mississauga, ON). DNA samples were treated with RNase (Fermentas, Mississauga, ON) to remove RNA contamination. DNA concentration and quality was assessed using Nanodrop 2000 (Thermo Scientific, Wilmington, Del.).

mtDNA Copy Number Analysis

Mitochondrial DNA copy number, relative to the diploid chromosomal DNA content, was quantitatively analyzed in tissues and cells using ABI 7300 real-time PCR (Applied Biosystems, CA). Primers were designed around COX-II region of the mitochondrial genome. Nuclear β-globin gene was used as a house-keeping gene.

### Oxygen Consumption Rate

Primary fibroblasts were plated at 2×105 cells per well. Oxygen consumption rates (pmol/min) were assessed using a XF Flux Analyzer (Seahorse Biosciences).

### MTT Assay for Cell Density

The colorimetric MTT (3-(4,5-dimethylthiazol-2-yl)-2,5-diphenyl tetrazolium bromide, tetrazolium) assay will be used to quantify the density of cells using manufacturer's instruction (Life Technologies, CA).

### Voluntary Activity Analyses

Animals were subjected to 3-day in-cage voluntary running wheel endurance exercise to assess their basal voluntary activity (Columbus Instruments). All mice were housed individually and had free access to food and water.

### Endurance Stress Test

The mice were subjected to endurance stress test to indirectly assess improvements in aerobic capacity with exercise. Mice were placed in individual lanes on the treadmill and allowed to acclimatize for 30 min to eliminate any confounding effects due to stress or anxiety related to a new environment. The test began with a 5-min warm-up session at 8 m/min, followed by 1-m/min increases in speed every 2 min until the mouse reached exhaustion. A low-intensity electrically stimulus was provided to ensure compliance. Time to exhaustion (in min) was recorded when the mouse sat at the lower end of the treadmill, near a shock bar, for >10 s and was unresponsive to further stimulation to continue running.

We claim:

1. An exosome pellet or physiological solution comprising exosomes, wherein the exosomes comprise one or more metabolic products.

2. The pellet or solution of claim 1, which is essentially free from undesirable entities having a diameter less than 20 nm and greater than 140 nm.

3. The pellet or solution of claim 1, comprising exosomal protein in an amount of about 100-2000 μg.

4. The pellet or solution of claim 1, wherein the metabolic product is a protein, mRNA or miRNA.

5. The pellet or solution of claim 4, wherein the metabolic product is selected from the group consisting of platelet-derived growth factor subunit B, meteorin-like protein, fibronectin type III domain-containing protein 5, fibronectin type III domain-containing protein 4, Shisa family member 5, secreted phosphoprotein 1, prolactin-inducible protein, tropomyosin alpha-1, proactivator polypeptide, vascular endothelial growth factor A, miR-677, miR-107, miR-133a-1, miR-496, miR-101b, miR-128-2, miR-469, miR-471, miR-15a, miR-679, miR-504, miR-411, miR-541, miR-707, miR-451, miR-125b-1, miR-690, miR-142, miR-219-2, miR-99b, miR-200b, miR-340, miR-551b, miR-101a and combinations thereof.

6. The pellet or solution of claim 1, wherein the exosomes are isolated from a biological sample from a mammal that regularly performs endurance exercise, wherein the sample is extracted from the mammal up to 4 hours post-exercise.

7. The pellet or solution of claim 6, wherein the exercise achieves a level of working of at least about 3-6 metabolic equivalents and is performed at least twice a week.

8. The pellet or solution of claim 5, wherein the biological sample is a blood sample.

9. The pellet or solution of claim 1, wherein the metabolic product is exogenous.

10. A method of inducing mitochondrial biogenesis in a mammal comprising administering to the mammal a physiological solution comprising resuspended exosomes as defined in claim 1.

11. The method of claim 10, wherein the exosomes are essentially free from undesirable entities having a diameter less than 20 nm and greater than 140 nm.

12. The method of claim 10, wherein the metabolic product is a protein, mRNA or miRNA.

13. The method of claim 12, wherein the metabolic product is selected from the group consisting of platelet-derived growth factor subunit B, meteorin-like protein, fibronectin type III domain-containing protein 5, fibronectin type III domain-containing protein 4, Shisa family member 5, secreted phosphoprotein 1, prolactin-inducible protein, tropomyosin alpha-1, proactivator polypeptide, vascular endothelial growth factor A, miR-677, miR-107, miR-133a-1, miR-496, miR-101b, miR-128-2, miR-469, miR-471, miR-15a, miR-679, miR-504, miR-411, miR-541, miR-707, miR-451, miR-125b-1, miR-690, miR-142, miR-219-2, miR-99b, miR-200b, miR-340, miR-551b, miR-101a and combinations thereof.

14. The method of claim 10, wherein the exosomes are isolated from a biological sample from a mammal that regularly performs endurance exercise, wherein the sample is extracted from the mammal up to 4 hours post-exercise.

15. The method of claim 14, wherein the exercise achieves a level of working of at least about 3-6 metabolic equivalents and is performed at least twice a week.

16. The method of claim 14, wherein the biological sample is a blood sample.

17. The method of claim 10, wherein the metabolic product is exogenous.

18. The method of claim 10, to treat metabolic syndrome, diseases of mitochondrial etiology, neuromuscular disease, neurometabolic disease, cancer, dementia, cardiovascular disease, cataracts, anemia, and infertility in the mammal.

19. The method of claim 18, wherein the metabolic syndrome is selected from the group consisting of obesity, metabolic syndrome, type 2 diabetes, non-alcohlic fatty liver disease, hyperinsulinemia, hypoinsulinemia, hypertension, hyperhepatosteatosis, hyperuricemia, fatty liver, polycystic ovarian syndrome, hyperphagia, acanthosis nigricans, endocrine abnormalities, triglyceride storage disease, Bardet-Biedl syndrome, Lawrence-Moon syndrome, Parder-Labhart-Willi syndrome, a primary mitochondrial genetic disorder, a neurological disease, and an age-associated pathology.
